(19) United States
(12) Patent Application Publication
(10) Pub. No.: US 2024/0310340 A1
(43) Pub. Date: Sep. 19, 2024

(54) SYSTEMS AND METHODS OF WATER TESTING WITH REAGENT PODS

(71) Applicant: ZODIAC POOL SYSTEMS LLC, Carlsbad, CA (US)

(72) Inventor: Minas Minassian, Carlsbad, CA (US)

(73) Assignee: ZODIAC POOL SYSTEMS LLC, Carlsbad, CA (US)

(21) Appl. No.: 18/604,996

(22) Filed: Mar. 14, 2024

Related U.S. Application Data
(60) Provisional application No. 63/452,887, filed on Mar. 17, 2023.

(51) Int. Cl. G01N 31/22; E04H 4/14; G01N 33/18

(52) U.S. Cl. CPC G01N 31/22; E04H 4/14; G01N 33/18

(57) ABSTRACT

A water testing pod for testing water of a swimming pool or spa includes a predefined volume of a predefined testing reagent encapsulated and sealed within a polymer membrane. A water testing system may include one or more of the water testing pods and a dispensing system, which may receive the one or more water testing pods and rupture the membrane for releasing the predetermined dosage of the testing reagent into the water of the swimming pool or spa. A method of testing water of a swimming pool or spa with a water testing system includes releasing a predetermined dosage of a testing reagent into the water of the swimming pool or spa by rupturing a membrane of a water testing pod.

### REFERENCE TO RELATED APPLICATION

This application claims the benefit of U.S. Provisional Patent Application No. 63/452,887, filed on Mar. 17, 2023, and entitled SYSTEMS AND METHODS FOR WATER TESTING WITH REAGENT PODS, the content of which is hereby incorporated by reference in its entirety.

### FIELD OF THE INVENTION

The invention relates to systems and methods for water testing of swimming pools and spas.

### BACKGROUND OF THE INVENTION

The water quality of water of water-containing vessels such as swimming pools and spas may change due to various factors such as debris, rain, sunlight, sweat, bacteria, etc., and as such swimming pools and spas require routine monitoring and adjustment of chemicals to control the quality of the water. Some pool owners may monitor and adjust the chemical levels themselves using testing reagent drops, but such testing reagents are often supplied in large volumes and it may be difficult for a pool owner to monitor and control dosage, with the volume being dispensed generally unknown or estimated by the pool owner. Moreover, because testing reagents are perishable and affected by temperature, air, etc., the useful lifetime of such testing reagents is limited, and a pool owner may have to discard unused testing reagents, thereby creating waste, or unknowingly use compromised testing reagents when trying to monitor and adjust water quality.

### SUMMARY

Embodiments covered by this patent are defined by the claims below, not this summary. This summary is a high-level overview of various embodiments and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to certain embodiments, a water testing pod for testing water of a swimming pool or spa includes a predefined volume of a predefined testing reagent encapsulated and sealed within a polymer membrane.

According to various embodiments water testing pod for testing water of a swimming pool or spa, the water testing pod comprising a predefined volume of a predefined testing reagent encapsulated and sealed within a polyamide or nylon-based membrane.

According to various embodiments, a water testing pod for testing water of a swimming pool or spa includes a predefined volume of a predefined testing reagent encapsulated and sealed within a water-soluble membrane.

According to some embodiments, a water testing system for testing water of a swimming pool or spa includes one or more water testing pods of any preceding or subsequent statement or combination of statements and a dispensing system configured to receive the one or more water testing pods and rupture the membrane for releasing the predetermined dosage of the testing reagent into the water of the swimming pool or spa.

According to various embodiments, a water testing system for testing water of a swimming pool or spa includes a plurality of water testing pods including at least a first water testing pod and a second water testing pod, and the first water testing pod and the second water testing pod each include a polymer membrane defining an internal volume. In certain embodiments, the first water testing pod includes a predetermined dosage of a first testing reagent sealed within the polymer membrane of the first water testing pod, and the second water testing pod comprises a predetermined dosage of a second testing reagent sealed within the polymer membrane of the second water testing pod. In some embodiments, a characteristic of the polymer membrane of the first water testing pod is different from a characteristic of the polymer membrane of the second water testing pod.

According to various embodiments, a water testing system for testing water of a swimming pool or spa includes one or more water testing pods, each water testing pod of the one or more water testing pods having a predetermined dosage of a testing reagent sealed within a polymer membrane. The system includes a dispensing system which may receive the one or more water testing pods and rupture the membrane for releasing the predetermined dosage of the testing reagent into the water of the swimming pool or spa.

According to some embodiments, a water testing system for testing water of a swimming pool or spa includes one or more water testing pods, each water testing pod of the one or more water testing pods having a predetermined dosage of a testing reagent sealed within a water-soluble membrane. In some embodiments, a dispensing system may receive the one or more water testing pods and rupture the membrane for releasing the predetermined dosage of the testing reagent into the water of the swimming pool or spa.

According to certain embodiments, a method of testing water of a swimming pool or spa with a water testing system includes releasing a predetermined dosage of a testing reagent into the water of the swimming pool or spa by rupturing a membrane of a water testing podVarious implementations described herein can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The specification makes reference to the following appended figure, in which use of like reference numerals is intended to illustrate like or analogous components.

FIG. 1 illustrates a water testing pod according to embodiments.

FIG. 2 illustrates the water testing pod of FIG. 1 with a testing reagent according to embodiments.

FIG. 3 illustrates a water testing system with the water testing pod of FIG. 1 according to embodiments.

### DESCRIPTION OF THE INVENTION

Described herein are water testing systems for testing water of swimming pools, spas, and other water-containing vessels. Compared to traditional water testing systems, the water testing systems described herein include one or more water testing pods having a single dosage (e.g., predetermined volume) of a testing reagent. The water testing pods include a membrane encapsulating the predetermined volume of the testing reagent, thereby sealing the testing reagent, and protecting the testing reagent from environmental exposure (e.g., air, temperature, etc.) that otherwise decreases the useful life of the testing reagents. As such, the testing reagent encapsulated within water testing pods have increased longevity compared to traditional approaches of supplying testing reagents for water testing. The water testing pods with the predetermined volume may provide improved testing of water as the pool owner is provided with a premeasured dosage. While a single dosage (e.g., single water testing pod) may be used, the water testing pods allow for improved calibration and/or ability to scale a dosage as needed by using any number of water testing pods as desired. The water testing pods may be dispensed into water of the swimming pool or spa using various dispensing systems or mechanisms as desired. In some embodiments, a dispensing system may include a release device that engages the water testing pod to rupture the membrane or otherwise empty the testing reagent from the water testing pod into the water. Additionally, or alternatively, the membrane of the water testing pod may be water soluble, and the water testing pods may be placed directly in the water to release the testing reagent. Various other benefits and advantages may be realized with the systems and methods provided herein, and the aforementioned advantages should not be considered limiting.

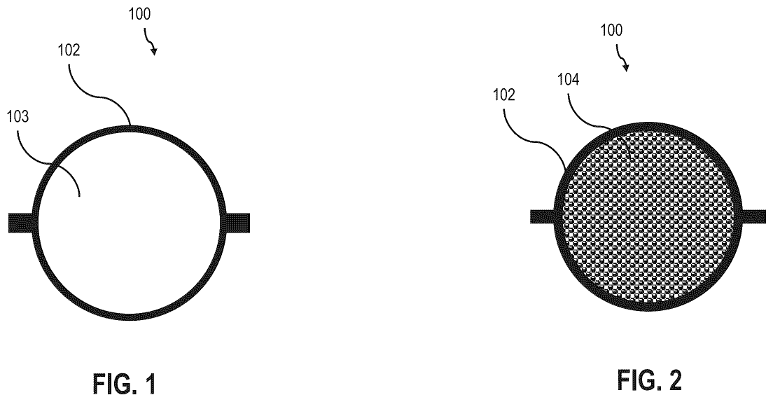

FIGS. 1 and 2 illustrate a water testing pod 100 for treating water of a swimming pool or spa according to embodiments. The water testing pod 100 generally includes a membrane 102 that defines an internal, predefined volume 103. As illustrated in FIG. 2, a testing reagent 104 is provided within the internal, predefined volume 103 such that the water testing pod 100 has a predefined volume of the testing reagent 104. While the membrane 102 is illustrated as forming the water testing pod 100 with a generally circular shape, the particular shape of the membrane 102 and/or the water testing pod 100 should not be considered limiting.

The membrane 102 may be constructed from various materials suitable for encapsulating and sealing the testing reagent 104 and protecting the testing reagent 104 from exposure to various environmental elements. In various embodiments, the membrane 102 may be constructed from materials such that the encapsulated testing reagent 104 does not leak during storage and/or maintains its integrity until ruptured. In some embodiments, the membrane 102 may be formed from a polymeric material, and optionally formed as a film material that may be further formed into the water testing pod 100. As non-limiting examples, the membrane 102 may be a film material that is obtained from a polymeric material by casting, blow-molding, extrusion, blow extrusion, combinations thereof, and/or other techniques as desired. Materials suitable for the membrane 102 may include, but are not limited to, polymers such as polyvinyl alcohols, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, maleic/acrylic acids, polysaccharides, natural gums, combinations thereof, and/or other materials as desired. Optionally, the membrane 102 may be a water-soluble material, although it need not be in other embodiments.

In certain embodiments, one or more characteristics of the membrane 102 may be controlled based on the particular testing reagent 104 to be encapsulated and/or protection to be provided to the testing reagent 104. As non-limiting examples, the type of material (or combination of materials) used for the membrane 102, the thickness of the membrane 102, the shape of the membrane 102 (and thus the shape of the water testing pod 100), combinations thereof, and/or other characteristics as desired, may be controlled based on the particular testing reagent 104. Optionally, when a plurality of testing reagents is provided to a pool owner or user in the form of the water testing pods 100, at least one characteristic of the membrane 102 and/or the water testing pod 100 may be varied for an improved visual identification and distinction between the various testing reagents. As non-limiting examples, a first type of testing reagent may be provided as a water testing pod 100 having a spherical or circular shape, and a second type of testing reagent may be provided as a water testing pod 100 having a spherical or circular shape with a greater diameter or size, a different shape (e.g., a triangular-shaped pod, disc-shaped pod, etc.), a different color, etc.

The testing reagent 104 may be various types of testing reagents as desired for testing water of the swimming pool or spa. As non-limiting examples, the testing reagent 104 may be reagents used for testing various water characteristics, such as but not limited to, pH, total alkalinity, calcium hardness, total dissolved solids, chlorine levels, bromine levels, combinations thereof, and/or other characteristics as desired.

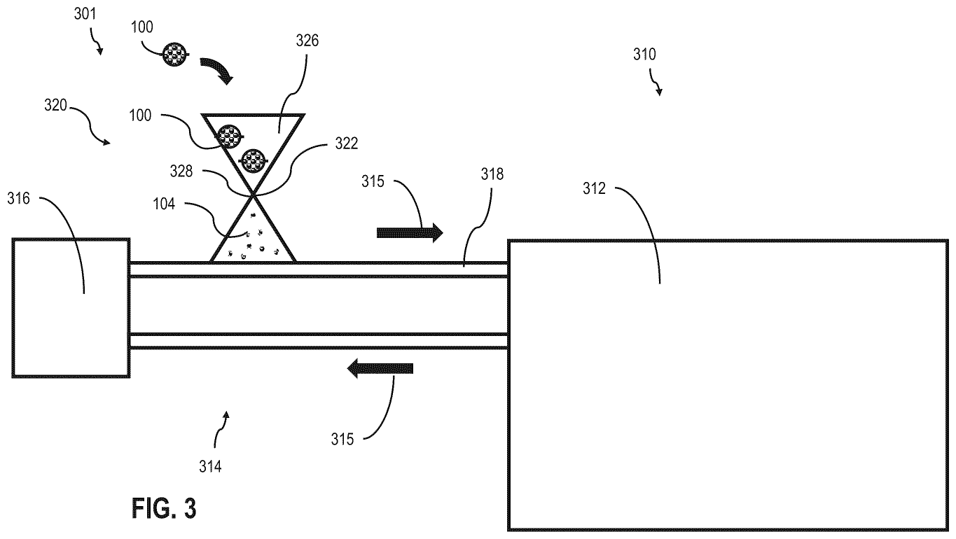

FIG. 3 illustrates a water testing system 301 for a pool system 310 and that utilizes one or more water testing pods 100. The water testing system 301 may test water of the pool system 310 causing the one or more water testing pods 100 to release the predefined volume of the testing reagent 104 into water of the pool system 310. In some embodiments, a single water testing pod 100 may be utilized; however, as mentioned, because the water testing pods 100 have the known, predetermined volume of the testing reagent 104, the pool owner may scale or calibrate the testing of the water by using as many water testing pods 100 as desired.

The one or more water testing pods 100 may release the testing reagent 104 into the water at various locations in the pool system 310, such as but not limited to, directly in a pool 312, in water flowing through a water circulation system 314 (water flow represented by arrows 315 in FIG. 3), and/or in other equipment and/or locations as desired. In the embodiment of FIG. 3, the water testing system 301 is illustrated as causing the one or more water testing pods 100 to release the testing reagent 104 in a return line 318 from a pump 316 of the water circulation system 314.

As mentioned, in some embodiments, the one or more water testing pods 100 may have a water-soluble membrane such that positioning the one or more water testing pods 100 within water of the pool system 310 causes the one or more water testing pods 100 to release the testing reagent 104. In some embodiments, regardless of the type of membrane 102, the water testing system 301 may include a dispensing system 320 that actively ruptures the membrane 102 to cause the one or more water testing pods 100 to release the predetermined dosage of the testing reagent 104. In such embodiments, the dispensing system 320 may include various devices or mechanisms for rupturing the membrane 102. As non-limiting examples, the dispensing system 320 may include a releasing mechanism or device 322 that ruptures the membrane 102 by applying a compressive force, puncturing the membrane 102, and/or otherwise compromising the membrane 102 such that the predetermined dosage of the testing reagent 104 is released. In the embodiment illustrated in FIG. 3, the dispensing system 320 includes a hopper 326 that receives one or more water testing pods 100, and the hopper 326 includes a crushing mechanism 328 as the releasing device 322 that crushes the one or more water testing pods 100 to release the testing reagent 104. The dispensing system 320 illustrated should not be considered limiting, and in other embodiments, the dispensing system 320 may include any number and/or type of releasing devices 322 and at various locations in the pool system 310 as desired.

Exemplary concepts or combinations of features of the invention may include:
A. A water testing pod for testing water of a swimming pool or spa, the water testing pod comprising a predefined volume of a predefined testing reagent encapsulated and sealed within a polymer membrane.
B. The water testing pod of any preceding or subsequent statement or combination of statements, wherein the polymer is selected from polyvinyl alcohols (PVA), polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, maleic/acrylic acids, polysaccharides, and/or natural gums or combinations thereof.
C. The water testing pod of any preceding or subsequent statement or combination of statements, wherein the polymer membrane is a polyamide membrane.
D. The water testing pod of any preceding or subsequent statement or combination of statements, wherein the polymer membrane is a nylon-based membrane.
E. The water testing pod of any preceding or subsequent statement or combination of statements, wherein the polymer membrane is a water-soluble material.
F. The water testing pod of any preceding or subsequent statement or combination of statements, wherein the testing reagent comprises at least one of a pH testing reagent, a total alkalinity testing reagent, a calcium hardness testing reagent, a total dissolved solids testing reagent, a chlorine level testing reagent, a bromine level testing reagent, or combinations thereof.
G. A water testing pod for testing water of a swimming pool or spa, the water testing pod comprising a predefined volume of a predefined testing reagent encapsulated and sealed within a polyamide or nylon-based membrane.
H. A water testing pod for testing water of a swimming pool or spa, the water testing pod comprising a predefined volume of a predefined testing reagent encapsulated and sealed within a water-soluble membrane.
I. A water testing system for testing water of a swimming pool or spa, the water testing system comprising:
   i. one or more water testing pods of any preceding or subsequent statement or combination of statements; and
   ii. a dispensing system configured to receive the one or more water testing pods and rupture the membrane for releasing the predetermined dosage of the testing reagent into the water of the swimming pool or spa.
J. The water testing system of any preceding or subsequent statement or combination of statements, wherein the at least one release system is configured to puncture the membrane.
K. The water testing system of any preceding or subsequent statement or combination of statements, wherein the dispensing system is configured to release the predetermined dosage of the testing reagent into the water of the swimming pool or spa flowing through a water circulation system of the swimming pool or spa.
L. A water testing system for testing water of a swimming pool or spa, the water testing system comprising:
   i. a plurality of water testing pods comprising at least a first water testing pod and a second water testing pod, wherein the first water testing pod and the second water testing pod each comprise a polymer membrane defining an internal volume,
   ii. wherein the first water testing pod comprises a predetermined dosage of a first testing reagent sealed within the polymer membrane of the first water testing pod,
   iii. wherein the second water testing pod comprises a predetermined dosage of a second testing reagent sealed within the polymer membrane of the second water testing pod, and
   iv. wherein a characteristic of the polymer membrane of the first water testing pod is different from a characteristic of the polymer membrane of the second water testing pod.
M. The water testing system of any preceding or subsequent statement or combination of statements, wherein the characteristic of the polymer member of the first water testing pod is a first shape and the characteristic of the polymer member of the second water testing pod is a second shape different from the first shape.
N. The water testing system of any preceding or subsequent statement or combination of statements, wherein the characteristic of the polymer member of the first water testing pod is a first transverse dimension and the characteristic of the polymer member of the second water testing pod is a second transverse dimension different from the first shape.
O. The water testing system of any preceding or subsequent statement or combination of statements, wherein the characteristic of the polymer member of the first water testing pod is a first material forming the polymer membrane and the characteristic of the polymer membrane of the second water testing pod is a second material forming the polymer membrane different from the first material.
P. The water testing system of any preceding or subsequent statement or combination of statements, wherein the characteristic of the polymer member of the first water testing pod is a first thickness of the polymer membrane and the characteristic of the polymer membrane of the second water testing pod is a second thickness of the polymer membrane different from the first thickness.
Q. The water testing system of any preceding or subsequent statement or combination of statements, further comprising a dispensing system configured to receive the plurality of water testing pods and rupture the membrane for releasing the predetermined dosages of the testing reagents into the water of the swimming pool or spa.
R. A water testing system for testing water of a swimming pool or spa, the water testing system comprising:
   i. one or more water testing pods, each water testing pod of the one or more water testing pods comprising a predetermined dosage of a testing reagent sealed within a polymer membrane; and
   ii. a dispensing system configured to receive the one or more water testing pods and rupture the membrane for releasing the predetermined dosage of the testing reagent into the water of the swimming pool or spa.
S. A water testing system for testing water of a swimming pool or spa, the water testing system comprising:
   i. one or more water testing pods, each water testing pod of the one or more water testing pods comprising a predetermined dosage of a testing reagent sealed within a water-soluble membrane; and
   ii. a dispensing system configured to receive the one or more water testing pods and rupture the membrane for releasing the predetermined dosage of the testing reagent into the water of the swimming pool or spa.
T. The water testing system of any preceding or subsequent statement or combination of statements, wherein the dispensing system comprises a hopper configured to receive the one or more water testing pods and at least one releasing device or mechanism for rupturing the membrane.
U. The water testing system of any preceding or subsequent statement or combination of statements, wherein the releasing device is configured to apply a compressing force on the membrane.
V. A method of testing water of a swimming pool or spa with a water testing system, the method comprising releasing a predetermined dosage of a testing reagent into the water of the swimming pool or spa by rupturing a membrane of a water testing pod, the water testing pod comprising the predetermined dosage of the testing agent.
W. The method of any preceding or subsequent statement or combination of statements, wherein rupturing the membrane of the water testing pod comprises compressing or puncturing the membrane.
X. The method of any preceding or subsequent statement or combination of statements, wherein the releasing of the predetermined dosage is into a water circulation system of the swimming pool or spa.

These examples are not intended to be mutually exclusive, exhaustive, or restrictive in any way, and the invention is not limited to these example embodiments but rather encompasses all possible modifications and variations within the scope of any claims ultimately drafted and issued in connection with the invention (and their equivalents). For avoidance of doubt, any combination of features not physically impossible or expressly identified as non-combinable herein may be within the scope of the invention. Further, although applicant has described devices and techniques for use principally with swimming pools and spas, persons skilled in the relevant field will recognize that the present invention conceivably could be employed in connection with other objects and in other manners. Finally, references to “pools” and “swimming pools” herein may also refer to spas or other water containing vessels used for recreation, training, or therapy and for which cleaning of debris is needed or desired.

The subject matter of embodiments of the present disclosure is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described. Directional references such as “up,” “down,” “top,” “bottom,” “left,” “right,” “vertical,” “horizontal,” “lateral,” “longitudinal,” “front,” and “back,” among others, are intended to refer to the orientation as illustrated and described in the figure (or figures) to which the components and directions are referencing.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, or gradients thereof, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention, and does not pose a limitation on the scope of the invention unless otherwise claimed. No language in the specification should be construed as indicating any non-claimed element as essential to the practice of the invention.

The above-described aspects are merely possible examples of implementations, merely set forth for a clear understanding of the principles of the present disclosure. Many variations and modifications can be made to the above-described embodiment(s) without departing substantially from the spirit and principles of the present disclosure. All such modifications and variations are intended to be included herein within the scope of the present disclosure, and all possible claims to individual aspects or combinations of elements or steps are intended to be supported by the present disclosure. Moreover, although specific terms are employed herein, as well as in the claims that follow, they are used only in a generic and descriptive sense, and not for the purposes of limiting the described embodiments, nor the claims that follow.

That which is claimed:

1. A water testing pod for testing water of a swimming pool or spa, the water testing pod comprising a predefined volume of a predefined testing reagent encapsulated and sealed within a polymer membrane.

2. The water testing pod of claim 1, wherein the polymer is selected from polyvinyl alcohols (PVA), polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, maleic/acrylic acids, polysaccharides, and/or natural gums or combinations thereof.

3. The water testing pod of claim 1, wherein the polymer membrane is a polyamide membrane.

4. The water testing pod of claim 1, wherein the polymer membrane is a nylon-based membrane.

5. The water testing pod of claim 1, wherein the polymer membrane is a water-soluble material.

6. The water testing pod of claim 1, wherein the testing reagent comprises at least one of a pH testing reagent, a total alkalinity testing reagent, a calcium hardness testing reagent, a total dissolved solids testing reagent, a chlorine level testing reagent, a bromine level testing reagent, or combinations thereof.

7. A water testing system for testing water of a swimming pool or spa, the water testing system comprising:
one or more water testing pods according to claim 1; and
a dispensing system configured to receive the one or more water testing pods and rupture the membrane for releasing the predetermined dosage of the testing reagent into the water of the swimming pool or spa.

8. The water testing system of claim 7, wherein the dispensing system comprises a hopper configured to receive the one or more water testing pods and at least one release system for rupturing the membrane.

9. The water testing system of claim 7, wherein the at least one release system is configured to apply a compressing force on the membrane.

10. The water testing system of claim 7, wherein the at least one release system is configured to puncture the membrane.

11. The water testing system of claim 7, wherein the dispensing system is configured to release the predetermined dosage of the testing reagent into the water of the swimming pool or spa flowing through a water circulation system of the swimming pool or spa.

12. A water testing system for testing water of a swimming pool or spa, the water testing system comprising:
a plurality of water testing pods comprising at least a first water testing pod and a second water testing pod, wherein the first water testing pod and the second water testing pod each comprise a polymer membrane defining an internal volume,
wherein the first water testing pod comprises a predetermined dosage of a first testing reagent sealed within the polymer membrane of the first water testing pod,
wherein the second water testing pod comprises a predetermined dosage of a second testing reagent sealed within the polymer membrane of the second water testing pod, and
wherein a characteristic of the polymer membrane of the first water testing pod is different from a characteristic of the polymer membrane of the second water testing pod.

13. The water testing system of claim 12, wherein the characteristic of the polymer member of the first water testing pod is a first shape and the characteristic of the polymer member of the second water testing pod is a second shape different from the first shape.

14. The water testing system of claim 12, wherein the characteristic of the polymer member of the first water testing pod is a first transverse dimension and the characteristic of the polymer member of the second water testing pod is a second transverse dimension different from the first shape.

15. The water testing system of claim 12, wherein the characteristic of the polymer member of the first water testing pod is a first material forming the polymer membrane and the characteristic of the polymer membrane of the second water testing pod is a second material forming the polymer membrane different from the first material.

16. The water testing system of claim 12, wherein the characteristic of the polymer member of the first water testing pod is a first thickness of the polymer membrane and the characteristic of the polymer membrane of the second water testing pod is a second thickness of the polymer membrane different from the first thickness.

17. The water testing system of claim 12, further comprising a dispensing system configured to receive the plurality of water testing pods and rupture the membrane for releasing the predetermined dosages of the testing reagents into the water of the swimming pool or spa.

18. A method of testing water of a swimming pool or spa with a water testing system, the method comprising releasing a predetermined dosage of a testing reagent into the water of the swimming pool or spa by rupturing a membrane of a water testing pod, the water testing pod comprising the predetermined dosage of the testing agent.

19. The method of claim 18, wherein rupturing the membrane of the water testing pod comprises compressing or puncturing the membrane.

20. The method of claim 18, wherein the releasing of the predetermined dosage is into a water circulation system of the swimming pool or spa.
